(19) United States
(12) Patent Application Publication
(10) Pub. No.: US 2023/0380598 A1
(43) Pub. Date: Nov. 30, 2023

(54) INTELLIGENT BED AND INTELLIGENT MONITORING SYSTEM USING SAME

(71) Applicant: Pegatron Corporation, Taipei City (TW)

(72) Inventor: Shun-Chieh CHANG, Taipei City (TW)

(21) Appl. No.: 18/191,077

(22) Filed: Mar. 28, 2023

(30) Foreign Application Priority Data
May 26, 2022 (TW) 111119666

(51) Int. Cl. A47C 17/86; H04R 3/00; G10L 15/02; H04R 1/40

(52) U.S. Cl. CPC A47C 17/86; H04R 3/005; G10L 15/02; H04R 1/406

(57) ABSTRACT

An intelligent bed includes a bed body, a plurality of sound receiving elements, a voiceprint database, and a controller. The plurality of sound receiving elements is surroundingly arranged around the bed body at intervals. The sound receiving elements each are configured to receive an audio signal from an audio source. The voiceprint database includes a plurality of contextual voiceprints. The controller is coupled to the sound receiving elements and the voiceprint database and configured to: extract a voiceprint of the audio signal; determine whether the voiceprint is consistent with one of the contextual voiceprints; and generate a status notification signal based on the voiceprint being consistent with the one of the contextual voiceprints.

### CROSS-REFERENCE TO RELATED APPLICATION

This application claims the priority benefit of Taiwan application serial no. 111119666, filed on May 26, 2022. The entirety of the above-mentioned patent application is hereby incorporated by reference herein and made a part of this specification.

### BACKGROUND

### Technical Field

The present disclosure relates to an intelligent bed and an intelligent monitoring system including the intelligent bed.

### Description of Related Art

Current beds on the market, such as baby cribs, are designed to allow children to sleep safely. The baby crib with a fence is generally purchased to prevent a child (a baby or an infant) from falling out of the bed. When the child is awake, a caregiver is required to notice a state of the child at regular intervals, or install a video camera in the child room to monitor the state of the child at all times. However, such a way of caring, which requires high attention, may easily lead to tiredness, and in case of inattention, it is impossible to find in time when the child is in danger.

### SUMMARY

In view of the above, the present disclosure provides an intelligent bed and an intelligent monitoring system using the same, so as to alleviate the above known problem. An embodiment of the present disclosure provides an intelligent bed. The intelligent bed includes a bed body, a plurality of sound receiving elements, a voiceprint database, and a controller. The sound receiving elements are surroundingly arranged around the bed body at intervals, and each configured to receive an audio signal from an audio source. The voiceprint database includes a plurality of contextual voiceprints. The controller is coupled to the sound receiving elements and the voiceprint database and configured to extract a voiceprint of the audio signal, determine whether the voiceprint is consistent with one of the contextual voiceprints, and generate a status notification signal based on the fact that the voiceprint is consistent with the one of the contextual voiceprints.

An embodiment of the present disclosure provides an intelligent monitoring system. The intelligent monitoring system includes a mobile communication device and an intelligent bed. The intelligent bed wirelessly communicates with the mobile communication device. The intelligent bed includes a bed body, a plurality of sound receiving elements, a voiceprint database, a wireless communication element, and a controller. The sound receiving elements are surroundingly arranged around the bed body and each configured to receive an audio signal from an audio source. The voiceprint database includes a plurality of contextual voiceprints. The controller is coupled to the sound receiving elements and the voiceprint database and configured to extract a voiceprint of the audio signal, determine whether the voiceprint is consistent with one of the contextual voiceprints, control the wireless communication element to transmit a status notification signal to the mobile communication device based on the fact that the voiceprint is consistent with the one of the contextual voiceprints, determine whether a distance between the mobile communication device and the bed body is less than a preset distance value, and stop transmitting the status notification signal to the mobile communication device based on the fact that the distance is less than the preset distance value.

The intelligent bed in the embodiment of the present disclosure can automatically determine whether the voiceprint of the audio source is consistent with a set context and generate the status notification signal accordingly to prompt a current state of the audio source. In addition, the intelligent monitoring system in the embodiment of the present disclosure may further determine whether to continue transmitting the status notification signal according to the distance between the mobile communication device and the intelligent bed.

In order to better understand the above and other aspects of the present disclosure, specific embodiments are listed and described below in detail with reference to the accompanying drawings.

### BRIEF DESCRIPTION OF THE DRAWING

FIG. 1 shows a functional block diagram of an intelligent monitoring system according to an embodiment of the present disclosure.

FIG. 2 shows a schematic diagram of an intelligent bed according to an embodiment of the present disclosure.

FIG. 3 shows a flowchart of an embodiment of an intelligent monitoring method of the intelligent monitoring system 1 in FIG. 1.

FIG. 4 shows a flowchart of another embodiment of an intelligent monitoring method of the intelligent monitoring system 1 in FIG. 1.

### DESCRIPTION OF THE EMBODIMENTS

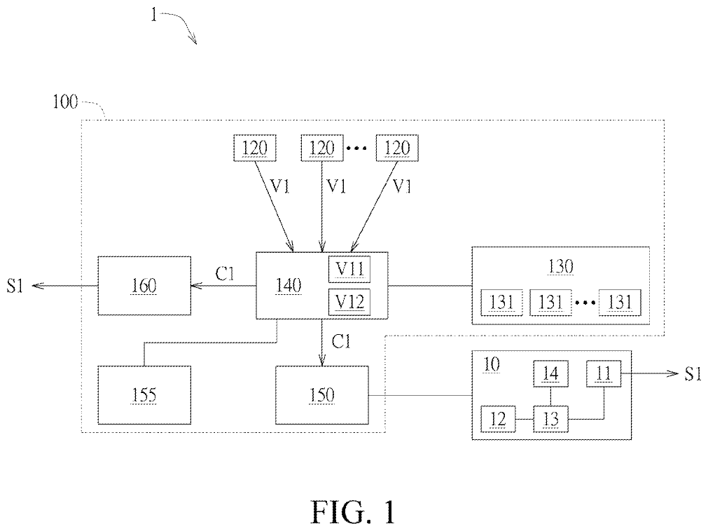

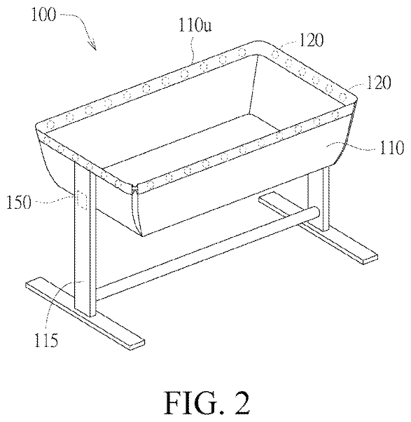

Referring to FIG. 1 and FIG. 2, FIG. 1 shows a functional block diagram of an intelligent monitoring system 10 according to an embodiment of the present disclosure, and FIG. 2 shows a schematic diagram of an intelligent bed 100 according to an embodiment of the present disclosure.

As shown in FIG. 1 and FIG. 2, the intelligent monitoring system 1 include an intelligent bed 100 and at least one mobile communication device 10. The intelligent bed 100 includes a bed body 110, a plurality of sound receiving elements 120, a voiceprint database 130 and a controller 140.

As shown in FIG. 2, the sound receiving elements 120 are surroundingly arranged around the bed body 110 at intervals, and each configured to receive an audio signal V1 from an audio source. In some embodiments, each of the sound receiving elements 120 is, for example, a microphone. The plurality of sound receiving elements 120 are arranged on an upper edge 110u of the bed body 110, and sound receiving surfaces or sound receiving holes of the sound receiving elements 120 are faced towards the bed body 110. Therefore, the audio signal V1 can be received by at least one of the sound receiving elements 120, regardless of a position of the audio source located on the bed body 110.

The voiceprint database 130 includes a plurality of contextual voiceprints 131. In some embodiments, the voiceprint database 130 may be configured in a storage element (such as a memory). The storage element is, for example, located in the controller 140, or configured outside the controller 140.

The controller 140 is coupled to the sound receiving elements 120 and the voiceprint database 130 and configured to extract a voiceprint V11 of the audio signal V1, compare the voiceprint V11 with the plurality of contextual voiceprints 131, and generate a status notification signal C1 according to a comparison result. In some embodiments, the controller 140 may be a digital signal processor, a plurality of microprocessors, one or more microprocessors that combine a core of the digital signal processor, a controller, a microcontroller, an application specific integrated circuit (ASIC), a field programmable gate array (FPGA), any other kind of integrated circuits, a state machine, a processor based on an advanced RISC machine (ARM), and the like.

As shown in FIG. 2, the intelligent bed 100 further includes a support 115. The support 115 is connected to the bed body 110. The bed body 110 and the support 115 are fixed to each other, or the bed body 110 may be pivotally connected to the support 115, so that the bed body 110 and the support 115 are rotatable relative to each other.

In the following description, the intelligent bed 100 is a baby crib as an example. The audio source is a baby, the audio signal V1 is a voice generated by the baby, and the voiceprint V11 is a voiceprint of the voice produced by the baby. However, the intelligent bed 100 in the embodiment of the present disclosure is not limited to the baby crib, which may be various bed products such as a pet bed, a hospital bed, and the like with monitoring requirements.

As shown in FIG. 1, based on the intelligent bed 100 as the baby crib, the contextual voiceprints 131 include at least one of a cry voiceprint, a turnover voiceprint, a milk regurgitation voiceprint, a sleep voiceprint, or a play voiceprint. However, the contextual voiceprints 131 may include more types of contextual voiceprints. In addition, the contextual voiceprint 131 may be obtained in advance before the intelligent bed 100 is used (or shipped). Alternatively, during use of the intelligent bed 100, the controller 140 may learn a new voiceprint of the audio signal V1, mark a context of the new voiceprint (a context name of the new voiceprint may be provided artificially), and then store the new contextual voiceprint in the voiceprint database 130. A quantity of contextual voiceprints 131 may be expanded through machine learning. In addition, the controller 140 may also expand the quantity of contextual voiceprints 131 in the voiceprint database 130 from a cloud server or a big data database.

In some embodiments, the intelligent bed 100 further includes a wireless communication element 150, a first positioning element 155, and/or a warning element 160. The controller 140, the wireless communication element 150, and/or the first positioning element 155 are, for example, circuits formed by using a semiconductor process. The wireless communication element 150 may be configured separately from the controller 140, or the wireless communication element 150 may be integrated into the controller 140. The first positioning element 155 may be configured separately from the controller 140, or may be integrated into the controller 140. In addition, the controller 140 is coupled to the voiceprint database 130, the wireless communication element 150, the first positioning element 155, and the warning element 160 to receive signals from the elements and/or to control the elements. In addition, at least one of the voiceprint database 130, the controller 140, the wireless communication element 150, the warning element 160, and the first positioning element 155 may be configured on the support 115 or the bed body 110.

As shown in FIG. 1, the wireless communication element 150 may communicate with the at least one mobile communication device 10. The wireless communication element 150 may transmit the status notification signal C1 to the mobile communication device 10. Specifically, the wireless communication element 150 is, for example, any communication module that conforms to a wireless communication protocol. The wireless communication protocol may be, for example, Bluetooth, the fifth-generation mobile communication technology, Wi-Fi, ZigBee, and the like. The first positioning element 155 is, for example, a global positioning system (GPS) module.

As shown in FIG. 1, the mobile communication device 10 further includes a wireless communication element 12, a second positioning element 14, and a controller 13. The wireless communication element 12 communicates with the wireless communication element 150 of the intelligent bed 100. The first positioning element 155 of the intelligent bed 100 is configured to obtain a first position of the bed body 110. The second positioning element 14 of the mobile communication device 10 is configured to obtain a second position of the mobile communication device 10. The mobile communication device 10 may transmit information about the second position to the intelligent bed 100, and/or the intelligent bed 100 may transmit information about the first position to the mobile communication device 10. The controller 13 of the mobile communication device 10 is electrically connected to the warning element 11, the wireless communication element 12, and the second positioning element 14, to control operation of the elements and receive signals of the elements. In addition, the wireless communication element 12 has a same or similar structure as the wireless communication element 150, the second positioning element 14 has a same or similar structure as the first positioning element 155, and the controller 13 has a same or similar structure as the controller 140. The details are not described herein again.

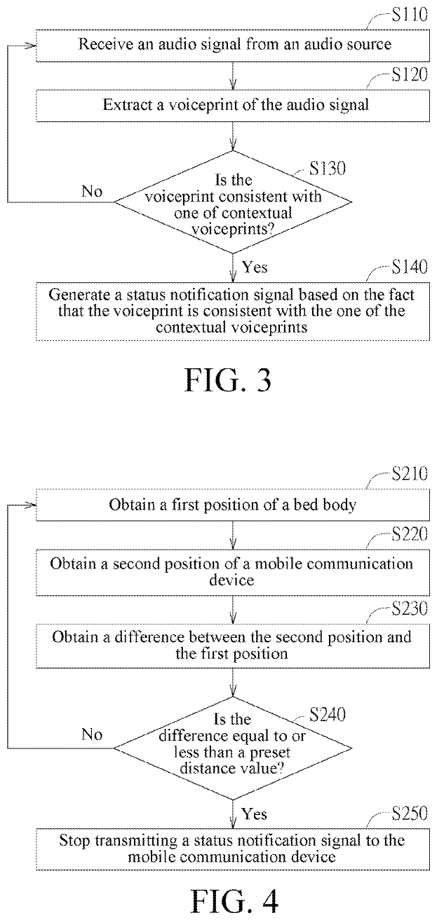

Referring to FIG. 1 and FIG. 3 together, FIG. 3 shows a flowchart of an embodiment of an intelligent monitoring method of the intelligent monitoring system 1 in FIG. 1.

In step S110, the sound receiving element 120 receives the audio signal V1 from the audio source. In an embodiment, the sound receiving element 120 receives a voice made by an infant as the audio signal V1.

Next, in step S120, the controller 140 extracts a voiceprint V11 of the audio signal V1. In an embodiment, the controller 140 extracts the voiceprint V11 of the audio signal V1 of the infant.

Next, in step S130, the controller 140 determines whether the voiceprint V11 is consistent with one of the plurality of contextual voiceprints 131 in the voiceprint database 130. If the voiceprint V11 is consistent with the one of the contextual voiceprints 131 in the voiceprint database 130, the process proceeds to step S140. If the voiceprint V11 is not one of the contextual voiceprints 131 in the voiceprint database 130, the process returns to step S110. In detail, the controller 140 determines whether the voiceprint V11 is a known voiceprint. If so, step S140 is performed, and further analysis is performed on the voiceprint V11. If not, it is determined that the voiceprint V11 may be noise, and step S110 is performed to continuously monitor whether the audio source produces a sound.

In step S140, the controller 140 generates the status notification signal C1 based on the fact that the voiceprint is consistent with the one of the contextual voiceprints 131.

In an embodiment, the controller 140 determines whether the voiceprint V11 is consistent with the cry voiceprint of the contextual voiceprints 131, and generates the status notification signal C1 based on the fact that the voiceprint V11 is consistent with the cry voiceprint and lasts for a period of time. The “period of time” is, for example, a number of seconds, such as 10 seconds. In another embodiment, the controller 140 determines whether the voiceprint V11 is consistent with the sleep voiceprint of the contextual voiceprints 131, and determines whether the voiceprint V11 stops and that the audio signal V1 is not received within a period of time based on the fact that the voiceprint V11 is consistent with the sleep voiceprint. The status notification signal C1 is generated based on the fact that the sleep voiceprint stops and the audio signal V1 is not received within the period of time. The “period of time” is, for example, a number of seconds, such as 3 seconds to 10 seconds, or may be shorter or longer.

In an embodiment, the controller 140 may control the warning element 160 to transmit a warning signal S1 according to the status notification signal C1. The warning signal S1 is, for example, various indicating signals such as a sound, colored light, vibration, and the like. When the warning signal S1 is the sound, the warning element 160 is, for example, a loudspeaker. When the warning signal S1 is the colored light, the warning element 160 is, for example, a light emitter. When the warning signal S1 is the vibration, the warning element 160 is, for example, a vibrator. In an embodiment, the warning signal S1 is, for example, a beep of dB, colored light flashing red and blue alternately, or a vibration having a same vibration intensity as that of the mobile device during ringing.

In another embodiment, the warning signal S1 may also be transmitted by the mobile communication device 10. For example, the mobile communication device 10 may include the warning element 11. The warning element 11 has the same or similar structure as the warning element 160, and the details are not described herein again. The controller 140 may transmit the status notification signal C1 to the mobile communication device 10, and the warning element 11 of the mobile communication device 10 transmits the warning signal S1 according to the status notification signal C1 to warn a holder of the mobile communication device 10.

Based on the above, when the state of the infant conforms to the set context, the intelligent bed 100 generates the status notification signal C1 to prompt a current (latest) state of the infant or to warn that the infant is in danger. In this way, by virtue of the status notification signal C1, the caregiver does not need to pay frequent attention to the child in the intelligent bed 100, and can find in time a case that the child in the intelligent bed 100 is in danger.

In addition, in other embodiments, the intelligent bed 100 may determine the position of the audio source on the bed body 110 according to a volume value V12 of the audio signal V1. For example, the controller 140 is configured to extract the volume value V12 of the audio signal V1 received by each sound receiving element 120, and determine the position of the audio source on the bed body 110 according to a distribution of the volume values V12. For example, the controller 140 uses the sound receiving element 120 having a largest volume value V12 as the position of the audio source on the bed body 110. Alternatively, the controller 140 considers a region having a plurality of sound receiving elements 120 with the largest volume value V12 as the position of the audio source on the bed body 110. In another embodiment, the controller 140 may also extract a signal-to-noise ratio of the audio signal V1 received by each sound receiving element 120, and determine the position of the audio source on the bed body 110 according to the distribution of the signal-to-noise ratios.

In other embodiments, the controller 140 is further configured to determine whether the position of the audio source is located on an edge of the bed body 110, and generate the status notification signal C1 based on the position of the audio source on the edge of the bed body 110 (the audio source may easily leave the bed body 110 or fall from the edge of the bed body 110). In an embodiment, a larger volume value V12 indicates that the audio source is increasingly closer to the edge of the bed body 110. The controller 140 may determine whether the volume value V12 exceeds a volume threshold value. If the volume value V12 exceeds the volume threshold value, it indicates that the audio source is already located on the edge of the bed body 110 and the status notification signal C1 is generated accordingly. The volume threshold value is, for example, more than 50 decibels. In another embodiment, the controller 140 may determine whether the signal-to-noise ratio exceeds a signal-to-noise ratio threshold. If the signal-to-noise ratio exceeds the signal-to-noise ratio threshold, it indicates that the audio source is already located on the edge of the bed body 110, and the status notification signal C1 is generated. The signal-to-noise ratio threshold is, for example, between 10 decibels and 25 decibels.

Referring to FIG. 1 and FIG. 4 together, FIG. 4 shows a flowchart of another embodiment of an intelligent monitoring method of the intelligent monitoring system 1 in FIG. 1. After the status notification signal C1 is transmitted to the at least one mobile communication device 10, the controller 140 can stop transmitting the status notification signal C1 to all of the mobile communication devices 10 as long as a distance between at least one of the at least one mobile communication device 10 and the bed body 110 is less than a preset distance value. For example, after the status notification signal C1 is transmitted to the plurality of mobile communication devices 10, the controller 140 determines whether the distance between each of the mobile communication devices 10 and the bed body 110 is less than the preset distance value, and the controller 140 stops transmitting the status notification signal C1 to the mobile communication devices 10 based on the fact that the distance between the one of the mobile communication devices 10 and the bed body 110 is less than the preset distance value. The process of FIG. 4 is further used as an example for description below.

In step S210, a first positioning element 155 of an intelligent bed 100 obtains a first position of a bed body 110. In an embodiment, the first position is a coordinate value.

In step S220, a second positioning element 14 of a mobile communication device 10 obtains a second position of the mobile communication device 10. In an embodiment, the second position is a coordinate value.

In step S230, a controller 140 calculates a distance between the mobile communication device 10 and the bed body 110 according to the first position and the second position. For example, the controller 140 performs differential operation on the second position and the first position to obtain a difference between the second position and the first position. The difference is the distance between the intelligent bed 100 and the mobile communication device 10.

In step S240, the controller 140 determines whether the difference is less than a preset distance value. The “preset distance value” is, for example, several centimeters, for example, less than 1 meter. When the difference is equal to or less than the preset distance value, the process proceeds to step S250. When the difference is not less than the preset distance value, the process returns to step S210.

In step S250, when the difference is equal to or less than the preset distance value, the controller 140 stops transmitting a status notification signal C1 to the mobile communication device 10. In this way, as long as the mobile communication device 10 moves next to the intelligent bed 100, the mobile communication device 10 can automatically cancel the notification signal without a need to manually cancel the notification signal. In addition, the mobile communication device 10 and/or the intelligent bed 100 may stop transmitting the warning signal S1 based on the fact that the status notification signal C1 stops being transmitted.

In practical application, the intelligent monitoring method in FIG. 4 may be applicable to an environment with a plurality of guardians and one ward. Each of the plurality of guardians has the mobile communication device 10, and the ward is an audio source located on the intelligent bed 100. In this embodiment, when the controller 140 of the intelligent bed 100 generates and transmits the status notification signal C1 to the mobile communication device 10 of each guardian. If one of the guardians moves next to the intelligent bed 100, that is to say, when a distance between the first position of the bed body 110 of the intelligent bed 100 and the second position of the mobile communication device 10 of the guardian is less than the preset distance value, the controller 140 of the intelligent bed 100 stops transmitting the status notification signal C1 to the mobile communication devices 10 of all of the guardians. In such an arrangement manner, when the ward is in need or in danger, only one of the guardians needs to go to a place where the ward is located for confirmation and assistance, and other guardians do not need to go to the place where the ward is located. In this way, the guardian can be prevented from wasting much unnecessary time in repeatedly confirming a condition of the ward.

In another embodiment, the difference may also be calculated by a controller 13 of the mobile communication device 10 (step S230). The mobile communication device 10 transmits the calculation result to the intelligent bed 100, and the intelligent bed 100 performs steps S240 and S250. Alternatively, steps S230 and S240 are performed by the controller 13 of the mobile communication device 10. The mobile communication device 10 transmits a determination result of step S240 to the intelligent bed 100, and the intelligent bed 100 performs step S250.

Based on the above, the embodiment of the present disclosure provides an intelligent bed. The intelligent bed includes a plurality of sound receiving elements that can receive an audio signal from an audio source. The controller may extract the voiceprint of the audio signal and compare the voiceprint with the plurality of contextual voiceprints. Based on the fact that the voiceprint is consistent with one of the contextual voiceprints, the status notification signal is generated, so as to prompt a current (latest) state of the audio source.

Based on the above, the present disclosure is disclosed above by using the embodiments, which are not intended to limit the present disclosure. Those with ordinary knowledge in the art to which the present disclosure belongs may make various changes and refinements without departing from the spirit and scope of the present disclosure. Therefore, the protection scope of the present disclosure shall be subject to the scope defined by the attached claims.

What is claimed is:

1. An intelligent bed, comprising:
a bed body;
a plurality of sound receiving elements, surroundingly arranged around the bed body at intervals and each configured to receive an audio signal from an audio source;
a voiceprint database, comprising a plurality of contextual voiceprints; and
a controller, coupled to the sound receiving elements and the voiceprint database and configured to:
extract a voiceprint of the audio signal;
determine whether the voiceprint is consistent with one of the contextual voiceprints; and
generate a status notification signal based on the voiceprint being consistent with the one of the contextual voiceprints.

2. The intelligent bed according to claim 1, wherein the sound receiving elements are arranged on an upper edge of the bed body.

3. The intelligent bed according to claim 1, wherein the controller is further configured to:
extract a volume value of the audio signal received by each of the sound receiving elements; and
determine a position of the audio source on the bed body according to a distribution of the volume values.

4. The intelligent bed according to claim 3, wherein the controller is further configured to:
determine whether the position is located at an edge of the bed body; and
generate the status notification signal based on the position being located at the edge of the bed body.

5. The intelligent bed according to claim 1, further comprising:
a wireless communication element, coupled to the controller and configured to transmit the status notification signal to a mobile communication device.

6. The intelligent bed according to claim 5, wherein the controller is further configured to:
determine whether a distance between the mobile communication device and the bed body is less than a preset distance value; and
stop transmitting the status notification signal to the mobile communication device based on the distance being less than the preset distance value.

7. The intelligent bed according to claim 1, further comprising a warning element, wherein the controller is further configured to:
control the warning element to transmit a warning signal according to the status notification signal.

8. The intelligent bed according to claim 1, wherein the contextual voiceprints comprise at least one of a cry voiceprint, a turnover voiceprint, a milk regurgitation voiceprint, a sleep voiceprint, or a play voiceprint.

9. The intelligent bed according to claim 1, wherein the controller is further configured to:
determine whether the voiceprint is consistent with a cry voiceprint of the contextual voiceprints; and
generate the status notification signal based on the voiceprint being consistent with the cry voiceprint and lasting for a period of time.

10. The intelligent bed according to claim 1, wherein the controller is further configured to:
determine whether the voiceprint is consistent with a sleep voiceprint of the contextual voiceprints;
determine whether the sleep voiceprint stops and stop receiving the audio signal based on the voiceprint being consistent with the sleep voiceprint; and
generate the status notification signal based on the sleep voiceprint stop and stop receiving the audio signal.

11. An intelligent monitoring system, comprising:
a mobile communication device; and
an intelligent bed, comprising:
a bed body;
a plurality of sound receiving elements, surroundingly arranged around the bed body and each configured to receive an audio signal from an audio source;
a voiceprint database, comprising a plurality of contextual voiceprints;
a wireless communication element, coupled to the mobile communication device; and
a controller, coupled to the sound receiving elements and the voiceprint database and configured to:
extract a voiceprint of the audio signal;
determine whether the voiceprint is consistent with one of the contextual voiceprints;
control the wireless communication element to transmit a status notification signal to the mobile communication device based on the voiceprint being consistent with the one of the contextual voiceprints;
determine whether a distance between the mobile communication device and the bed body is less than a preset distance value; and
stop transmitting the status notification signal to the mobile communication device based on the distance being less than the preset distance value.

12. The intelligent monitoring system according to claim 11, wherein the intelligent bed further comprises:
a first positioning element, configured to obtain a first position of the bed body, wherein
the mobile communication device further comprises a second positioning element, the second positioning element is configured to obtain a second position of the mobile communication device, and the controller is further configured to calculate the distance according to the first position and the second position.

13. The intelligent monitoring system according to claim 11, comprising a plurality of mobile communication devices, wherein the controller is further configured to:
determine whether a distance between each of the mobile communication devices and the bed body is less than a preset distance value; and
stop transmitting the status notification signal to the mobile communication devices based on the distance between the one of the mobile communication devices and the bed body is less than the preset distance value.
